(19) United States
(12) Patent Application Publication
(10) Pub. No.: US 2018/0140593 A1
(43) Pub. Date: May 24, 2018

(54) Treatment of Glaucoma Using Laquinimod

(71) Applicants: Ron Neumann, Ramat Hasharon (IL); Revital Etzyoni, Kfar-Saba (IL)

(72) Inventors: Ron Neumann, Ramat Hasharon (IL); Revital Etzyoni, Kfar-Saba (IL)

(73) Assignee: Teva Pharmaceutical Industries, Ltd., Petach-Tikva (IL)

(21) Appl. No.: 15/875,833

(22) Filed: Jan. 19, 2018

Related U.S. Application Data
(63) Continuation of application No. 15/701,168, filed on Sep. 11, 2017.
Application No. 15/701,168 is a continuation of application No. 15/611,578, filed on Jun. 1, 2017, now abandoned.
Application No. 15/611,578 is a continuation of application No. 15/414,406, filed on Jan. 24, 2017, now abandoned.
Application No. 15/414,406 is a continuation of application No. 15/290,461, filed on Oct. 11, 2016, now abandoned.
Application No. 15/290,461 is a continuation of application No. 14/540,768, filed on Nov. 13, 2014, now abandoned.
(60) Provisional application No. 61/904,962, filed on Nov. 15, 2013.

(51) Int. Cl. A61K 31/4704; A61K 9/20; A61K 45/06; A61K 9/00; A61J 1/03; A61K 9/28; B65D 1/02; B65D 81/26

(52) U.S. Cl. CPC A61K 31/4704; A61K 9/2054; A61K 45/06; A61K 9/0048; A61K 9/2013; A61J 1/035; A61K 9/2018; A61K 9/28; B65D 1/0207; B65D 81/266; A61K 9/0053; A61K 9/2009

(57) ABSTRACT

The subject invention provides a method of treating a subject afflicted with glaucoma, suffering from retinal ganglion cell (RGC) loss or damage, or elevated intraocular pressure (IOP), or of reducing RGC loss or damage, or reducing IOP in a subject, comprising administering to the subject an amount of laquinimod effective to treat the subject, to reduce RGC loss or damage, or to reduce IOP in the subject. Provide also is a pharmaceutical composition, a package and a therapeutic package for treating a subject afflicted with glaucoma.

This application claims benefit of U.S. Provisional Application No. 61/904,962, filed Nov. 15, 2013, the entire content of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

Glaucoma is a group of ocular diseases characterized by progressive damage to the eye at least partly due to elevated intraocular pressure (IOP) (Merck Manual of Diagnosis and Therapy (1999)). Additionally, glaucoma is characterized by retinal ganglion cell (RGC) death, axon loss and an excavated appearance of the optic nerve head (Alward 1998). Glaucoma can be diagnosed before vision loss occurs by visual field testing and by ophthalmoscopic examination of the optic nerve to detect “cupping.” The mean IOP in normal adults is 15 to 16 mm Hg; the normal range is 10 to 21 mm Hg. One form of management of glaucoma is based on lowering the IOP using topically applied medications (Coleman 1999).

Currently there are five major classes of medications that are used to lower the IOP: β-adrenergic antagonists, adrenergic agonists, parasympathomimetics, prostaglandin-like analogues and carbonic anhydrase inhibitors (Medeiros et al. 2002). Although most medications are applied topically to the eye, they can cause severe systemic side effects and adversely affect the quality of the patient's life. If additional lowering of IOP is indicated or if medication fails to sufficiently lower the IOP, laser trabeculoplasty is usually the next step. If IOP is still not adequately controlled, incisional glaucoma surgery is indicated (Id). The lowering of IOP, despite significantly reducing the extent of neuronal loss, does not ensure cessation of the disease process, because the loss of RGCs may continue. Recent studies of the association between IOP regulation and visual field loss after medical or surgical intervention showed that ongoing neuronal loss reflected in visual field tests can be diminished if the IOP is low. However, neuronal loss may continue to occur after reduction of IOP (Bakalash et al. 2002).

Glaucomatous optic neuropathy appears to result from specific pathophysiological changes and subsequent death of RGCs and their axons. The process of RGC death is thought to be biphasic: a primary injury responsible for initiation of damage followed by a slower, secondary degeneration attributable to the hostile environment surrounding the degenerating cells (Kipnis et al. 2000).

The molecular mechanism triggering RGC death has not been identified. Deprivation of neurotrophic factors, ischemia, chronic elevation of glutamate and disorganized nitric oxide metabolism are suspected to be possible mechanisms (Farkas et al. 2001). In addition, it is possible that the mechanisms leading to RGC death share common features with other types of neuronal injury, such as signaling by reactive oxygen species, depolarization of mitochondria, or induction of transcriptionally regulated cell death (Weinreb et al. 1999).

Laquinimod

Laquinimod (LAQ) is a novel synthetic compound with high oral bioavailability which has been suggested as an oral formulation for the treatment of Multiple Sclerosis (MS) (Polman, 2005; Sandberg-Wollheim, 2005). Laquinimod and its sodium salt form are described, for example, in U.S. Pat. No. 6,077,851. The mechanism of action of laquinimod is not fully understood. Animal studies show it causes a Th1 (T helper 1 cell, which produces pro-inflammatory cytokines) to Th2 (T helper 2 cell, which produces anti-inflammatory cytokines) shift with an anti-inflammatory profile (Yang, 2004; Brück, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Brück, 2011).

The effects of laquinimod on glaucoma have not previously been studied.

### SUMMARY OF THE INVENTION

The subject invention provides a method of treating a subject afflicted with glaucoma comprising administering to the subject an amount of laquinimod effective to treat the subject.

The subject invention also provides a method of treating a subject suffering from retinal ganglion cell loss or retinal ganglion cell damage, or of reducing retinal ganglion cell loss or damage in a subject, comprising administering to the subject an amount of laquinimod effective to reduce retinal ganglion cell loss or retinal ganglion cell damage in the subject.

The subject invention also provides a method of treating a subject suffering from elevated intraocular pressure, or of reducing intraocular pressure in a subject, comprising administering to the subject an amount of laquinimod effective to reduce intraocular pressure in the subject.

The subject invention also provides a package comprising a) a pharmaceutical composition comprising an amount of laquinimod; and b) instruction for use of the pharmaceutical composition to treat a subject afflicted with glaucoma.

The subject invention also provides a therapeutic package for dispensing to, or for use in dispensing to, a subject afflicted with glaucoma, which comprises a) one or more unit doses, each such unit dose comprising an amount of laquinimod thereof, wherein the amount of said laquinimod in said unit dose is effective, upon administration to said subject, to treat the subject, and b) a finished pharmaceutical container therefor, said container containing said unit dose or unit doses, said container further containing or comprising labeling directing the use of said package in the treatment of said subject.

The subject invention also provides a pharmaceutical composition and a package as described herein for use in treating a subject afflicted with glaucoma.

The subject invention also provides a pharmaceutical composition in unit dosage form, useful in treating a subject afflicted with glaucoma, which comprises an amount of laquinimod; which amount of said laquinimod in said composition is effective, upon administration to said subject of one or more of said unit dosage forms of said composition, to treat the subject.

The subject invention also provides a package comprising a) a pharmaceutical composition as described herein; and b) instruction for use of the pharmaceutical composition to treat a subject afflicted with glaucoma.

The subject invention also provides laquinimod for the manufacture of a medicament for use in treating a subject afflicted glaucoma.

### BRIEF DESCRIPTION OF THE FIGURES

FIG. 1: Example 1: Mean ΔIOP (OHT minus Non-OHT) (mmHg).

FIG. 2: Example 1: % Fluoro-gold Labeled RGC Loss.

FIG. 3: Example 1: Mean Fluoro-gold Labeled RGC count per mm².

FIG. 4: Optic Nerve Injury Grade (1-5).

FIG. 5A: Example 1: Representative images of the retinas with FG-labeled RGC—Animal Number 48, Group 1, Left Eye.

FIG. 5B: Example 1: Representative images of the retinas with FG-labeled RGC—Animal Number 48, Group 1, Right Eye.

FIG. 5C: Example 1: Representative images of the retinas with FG-labeled RGC—Animal Number 59, Group 2, Left Eye.

FIG. 5D: Example 1: Representative images of the retinas with FG-labeled RGC—Animal Number 59, Group 2, Right Eye.

FIG. 5E: Example 1: Representative images of the retinas with FG-labeled RGC—Animal Number 22, Group 3, Left Eye.

FIG. 5F: Example 1: Representative images of the retinas with FG-labeled RGC—Animal Number 22, Group 3, Right Eye.

FIG. 5G: Example 1: Representative images of the retinas with FG-labeled RGC—Animal Number 31, Group 4, Left Eye.

FIG. 5H: Example 1: Representative images of the retinas with FG-labeled RGC—Animal Number 31, Group 4, Right Eye.

FIG. 5I: Example 1: Representative images of the retinas with FG-labeled RGC—Animal Number 35, Group 5, Left Eye.

FIG. 5J: Example 1: Representative images of the retinas with FG-labeled RGC—Animal Number 35, Group 5, Right Eye.

### DETAILED DESCRIPTION OF THE INVENTION

The subject invention provides a method of treating a subject afflicted with glaucoma comprising administering to the subject an amount of laquinimod effective to treat the subject.

In one embodiment, the administration of laquinimod is effective to reduce or inhibit a symptom of the glaucoma in the subject. In another embodiment, the symptom is retinal ganglion cell damage, retinal ganglion cell loss, or elevated intraocular pressure. In another embodiment, laquinimod is laquinimod sodium.

In one embodiment, the route of administration of laquinimod is intraocular, periocular, systemic or topical. In another embodiment, laquinimod is administered via oral administration. In another embodiment, laquinimod is administered via ocular administration. In another embodiment, laquinimod is administered in the form of an aerosol, an inhalable powder, an injectable, a liquid, a gel, a solid, a capsule or a tablet.

In an embodiment, the concentration of laquinimod in the liquid or gel is 5-100 mg/ml solution, 20-100 mg/ml solution, 10-15 mg/ml solution, or 20-50 mg/ml solution. In another embodiment, laquinimod is administered periodically. In another embodiment, laquinimod is administered daily. In another embodiment, laquinimod is administered more often than once daily. In yet another embodiment, laquinimod is administered less often than once daily.

In one embodiment, the amount laquinimod administered is at least 0.2 mg/day and/or less than 0.6 mg/day. In another embodiment, the amount laquinimod administered is 0.03-600 mg/day, 0.1-40.0 mg/day, 0.1-2.5 mg/day, 0.25-2.0 mg/day or 0.5-1.2 mg/day. In another embodiment, the amount laquinimod administered is 0.25 mg/day, 0.3 mg/day, 0.5 mg/day, 0.6 mg/day, 1.0 mg/day, 1.2 mg/day, 1.5 mg/day or 2.0 mg/day. In yet another embodiment, the amount of laquinimod administered is 0.05-4.0 mg per administration, 0.05-2.0 mg per administration, 0.2-4.0 mg per administration, 0.2-2.0 mg per administration, about 0.1 mg per administration, or about 0.5 mg per administration.

In one embodiment of the present invention, the method further comprises administration of a second agent for the treatment of glaucoma. In another embodiment, the second agent is a β-adrenergic antagonist, adrenergic agonist, parasympathomimetic, prostaglandin-like analog, or carbonic anhydrase inhibitor.

In one embodiment, the periodic administration of laquinimod continues for at least 3 days, more than 30 days, more than 42 days, 8 weeks or more, at least 12 weeks, at least 24 weeks, more than 24 weeks, or 6 months or more. In another embodiment, the subject is a human patient.

The subject invention also provides a method of treating a subject suffering from retinal ganglion cell loss or retinal ganglion cell damage, or of reducing retinal ganglion cell loss or damage in a subject, comprising administering to the subject an amount of laquinimod effective to reduce retinal ganglion cell loss or retinal ganglion cell damage in the subject.

The subject invention also provides a method of treating a subject suffering from elevated intraocular pressure, or of reducing intraocular pressure in a subject, comprising administering to the subject an amount of laquinimod effective to reduce intraocular pressure in the subject.

The subject invention also provides a package comprising a) a pharmaceutical composition comprising an amount of laquinimod; and b) instruction for use of the pharmaceutical composition to treat a subject afflicted with glaucoma.

In one embodiment of the present invention, the package comprises a second pharmaceutical composition comprising an amount of a second agent for the treatment of glaucoma. In another embodiment, the second agent is a β-adrenergic antagonist, adrenergic agonist, parasympathomimetic, prostaglandin-like analog, or carbonic anhydrase inhibitor.

In one embodiment, the pharmaceutical composition is the form of an aerosol, an inhalable powder, an injectable, a liquid, a gel, a solid, a capsule or a tablet. In another embodiment, the pharmaceutical composition is in a liquid or a gel form.

In one embodiment, the concentration of laquinimod in the liquid or gel is 5-100 mg/ml solution, 20-100 mg/ml solution, 10-15 mg/ml solution or 20-50 mg/ml solution.

In another embodiment, the pharmaceutical composition is in capsule form or in tablet form. In another embodiment, the tablets are coated with a coating which inhibits oxygen from contacting the core. In another embodiment, the coating comprises a cellulosic polymer, a detackifier, a gloss enhancer, or pigment.

In one embodiment, the pharmaceutical composition further comprises mannitol. In another embodiment, the pharmaceutical composition further comprises an alkalinizing agent. In another embodiment, the alkalinizing agent is meglumine. In another embodiment, the pharmaceutical composition further comprises an oxidation reducing agent.

In one embodiment, the pharmaceutical composition is stable and free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In another embodiment, the lubricant is present in the pharmaceutical composition as solid particles. In another embodiment, the lubricant is sodium stearyl fumarate or magnesium stearate.

In one embodiment, the pharmaceutical composition further comprises a filler. In another embodiment, the filler is present in the pharmaceutical composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the package further comprises a desiccant. In another embodiment, the desiccant is silica gel.

In one embodiment, the pharmaceutical composition is stable and has a moisture content of no more than 4%. In another embodiment, laquinimod is present in the pharmaceutical composition as solid particles. In another embodiment, the package is a sealed packaging having a moisture permeability of not more than 15 mg/day per liter. In another embodiment, the sealed package is a blister pack in which the maximum moisture permeability is no more than 0.005 mg/day. In another embodiment, the sealed package is a bottle. In another embodiment, the bottle is closed with a heat induction liner. In another embodiment, the sealed package comprises an HDPE bottle. In another embodiment, the sealed package comprises an oxygen absorbing agent. In another embodiment, the oxygen absorbing agent is iron.

In one embodiment, the amount of laquinimod in the pharmaceutical composition is at least 0.2 mg or less than 0.6 mg. In another embodiment, the amount of laquinimod in the pharmaceutical composition is 0.1-40.0 mg, 0.03-600 mg, 0.1-2.5 mg, 0.25-2.0 mg, 0.5-1.2 mg, 0.25 mg, 0.3 mg, 0.5 mg, 0.6 mg, 1.0 mg, 1.2 mg, 1.5 mg, or 2.0 mg. In another embodiment, the pharmaceutical composition comprises unit doses of laquinimod of 0.05-4.0 mg, 0.05-2.0 mg, 0.2-4.0 mg, 0.2-2.0 mg, about 0.1 mg, or about 0.5 mg.

In one embodiment, the pharmaceutical composition is formulated for intraocular, periocular, systemic or topical administration. In another embodiment, the pharmaceutical composition is formulated for oral or ocular administration.

The subject invention also provides packages as described herein for use in treating a subject afflicted with glaucoma.

The subject invention also provides a therapeutic package for dispensing to, or for use in dispensing to, a subject afflicted with glaucoma, which comprises a) one or more unit doses, each such unit dose comprising an amount of laquinimod thereof, wherein the amount of said laquinimod in said unit dose is effective, upon administration to said subject, to treat the subject, and b) a finished pharmaceutical container therefor, said container containing said unit dose or unit doses, said container further containing or comprising labeling directing the use of said package in the treatment of said subject.

In one embodiment, the therapeutic package comprises a second pharmaceutical composition comprising an amount of a second agent for the treatment of glaucoma. In another embodiment, the second agent is a β-adrenergic antagonist, adrenergic agonist, parasympathomimetic, prostaglandin-like analog, or carbonic anhydrase inhibitor.

The subject invention also provides a pharmaceutical composition comprising an amount of laquinimod for use in treating a subject afflicted glaucoma.

In one embodiment, the pharmaceutical composition comprises an amount of a second agent for the treatment of glaucoma. In another embodiment, the second agent is a β-adrenergic antagonist, adrenergic agonist, parasympathomimetic, prostaglandin-like analog, or carbonic anhydrase inhibitor.

In an embodiment, the pharmaceutical composition is in the form of an aerosol, an inhalable powder, an injectable, a liquid, a gel, a solid, a capsule or a tablet. In another embodiment, the pharmaceutical composition is in a liquid or a gel form.

In an embodiment, the concentration of laquinimod in the liquid or gel is 5-100 mg/ml solution, 20-100 mg/ml solution, 10-15 mg/ml solution or 20-50 mg/ml solution. In another embodiment, the pharmaceutical composition comprises a unit dose of 10 μL of an aqueous pharmaceutical solution which contains in solution at least 0.2 mg laquinimod. In another embodiment, laquinimod is laquinimod sodium.

In one embodiment, the pharmaceutical composition is in capsule form or in tablet form. In another embodiment, the tablets are coated with a coating which inhibits oxygen from contacting the core. In another embodiment, the coating comprises a cellulosic polymer, a detackifier, a gloss enhancer, or pigment. In another embodiment, the pharmaceutical composition further comprises mannitol.

In one embodiment, the pharmaceutical composition further comprises an alkalinizing agent. In another embodiment, the alkalinizing agent is meglumine. In another embodiment, the pharmaceutical composition further comprises an oxidation reducing agent.

In one embodiment, the pharmaceutical composition is free of an alkalinizing agent or an oxidation reducing agent. In another embodiment, the pharmaceutical composition is free of an alkalinizing agent and free of an oxidation reducing agent.

In one embodiment, the pharmaceutical composition is stable and free of disintegrant. In another embodiment, the pharmaceutical composition further comprises a lubricant. In another embodiment, the lubricant is present in the pharmaceutical composition as solid particles. In another embodiment, the lubricant is sodium stearyl fumarate or magnesium stearate.

In one embodiment, the pharmaceutical composition further comprises a filler. In another embodiment, the filler is present in the pharmaceutical composition as solid particles. In another embodiment, the filler is lactose, lactose monohydrate, starch, isomalt, mannitol, sodium starch glycolate, sorbitol, lactose spray dried, lactose anhydrouse, or a combination thereof. In another embodiment, the filler is mannitol or lactose monohydrate.

In one embodiment, the amount of laquinimod in the pharmaceutical composition is at least 0.2 mg or less than 0.6 mg. In another embodiment, the amount of laquinimod in is 0.1-40.0 mg, 0.03-600 mg, 0.1-2.5 mg, 0.25-2.0 mg, 0.5-1.2 mg, 0.25 mg, 0.3 mg, 0.5 mg, 0.6 mg, 1.0 mg, 1.2 mg, 1.5 mg, or 2.0 mg. In another embodiment, the pharmaceutical composition comprises unit doses of laquinimod of 0.05-4.0 mg, 0.05-2.0 mg, 0.2-4.0 mg, 0.2-2.0 mg, about 0.1 mg, or about 0.5 mg.

In one embodiment, the pharmaceutical composition is formulated for intraocular, periocular, systemic or topical administration. In another embodiment, the pharmaceutical composition is formulated for oral or ocular administration.

The subject invention also provides a pharmaceutical composition as described herein for use in treating a subject afflicted with glaucoma.

The subject invention also provides a pharmaceutical composition in unit dosage form, useful in treating a subject afflicted with glaucoma, which comprises an amount of laquinimod; which amount of said laquinimod in said composition is effective, upon administration to said subject of one or more of said unit dosage forms of said composition, to treat the subject.

The subject invention also provides a package comprising a) a pharmaceutical composition as described herein; and b) instruction for use of the pharmaceutical composition to treat a subject afflicted with glaucoma.

The subject invention also provides laquinimod for the manufacture of a medicament for use in treating a subject afflicted glaucoma.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

### Terms

As used herein, and unless stated otherwise, each of the following terms shall have the definition set forth below.

As used herein, “laquinimod” means laquinimod acid or a pharmaceutically acceptable salt thereof.

A “salt thereof” is a salt of the instant compounds which have been modified by making acid or base salts of the compounds. The term “pharmaceutically acceptable salt” in this respect, refers to the relatively non-toxic, inorganic and organic acid or base addition salts of compounds of the present invention. For example, one means of preparing such a salt is by treating a compound of the present invention with an inorganic base.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion.

As used herein, a “unit dose”, “unit doses” and “unit dosage form(s)” mean a single drug administration entity/entities.

As used herein, “about” in the context of a numerical value or range means±10% of the numerical value or range recited or claimed.

As used herein, a composition that is “free” of a chemical entity means that the composition contains, if at all, an amount of the chemical entity which cannot be avoided although the chemical entity is not part of the formulation and was not affirmatively added during any part of the manufacturing process. For example, a composition which is “free” of an alkalizing agent means that the alkalizing agent, if present at all, is a minority component of the composition by weight. Preferably, when a composition is “free” of a component, the composition comprises less than 0.1 wt %, 0.05 wt %, 0.02 wt %, or 0.01 wt % of the component.

As used herein, “alkalizing agent” is used interchangeably with the term “alkaline-reacting component” or “alkaline agent” and refers to any pharmaceutically acceptable excipient which neutralizes protons in, and raises the pH of, the pharmaceutical composition in which it is used.

As used herein, “oxidation reducing agent” refers to a group of chemicals which includes an “antioxidant”, a “reduction agent” and a “chelating agent”.

As used herein, “antioxidant” refers to a compound selected from the group consisting of tocopherol, methionine, glutathione, tocotrienol, dimethyl glycine, betaine, butylated hydroxyanisole, butylated hydroxytoluene, turmerin, vitamin E, ascorbyl palmitate, tocopherol, deteroxime mesylate, methyl paraben, ethyl paraben, butylated hydroxyanisole, butylated hydroxytoluene, propyl gallate, sodium or potassium metabisulfite, sodium or potassium sulfite, alpha tocopherol or derivatives thereof, sodium ascorbate, disodium edentate, BHA (butylated hydroxyanisole), a pharmaceutically acceptable salt or ester of the mentioned compounds, and mixtures thereof.

The term “antioxidant” as used herein also refers to Flavonoids such as those selected from the group of quercetin, morin, naringenin and hesperetin, taxifolin, afzelin, quercitrin, myricitrin, genistein, apigenin and biochanin A, flavone, flavopiridol, isoflavonoids such as the soy isoflavonoid, genistein, catechins such as the tea catechin epigallocatechin gallate, flavonol, epicatechin, hesperetin, chrysin, diosmin, hesperidin, luteolin, and rutin.

As used herein, “reduction agent” refers to a compound selected from the group consisting of thiol-containing compound, thioglycerol, mercaptoethanol, thioglycol, thiodiglycol, cysteine, thioglucose, dithiothreitol (DTT), dithio-bis-maleimidoethane (DTME), 2,6-di-tert-butyl-4-methylphenol (BHT), sodium dithionite, sodium bisulphite, formamidine sodium metabisulphite, and ammonium bisulphite.”

As used herein, “chelating agent” refers to a compound selected from the group consisting of penicillamine, trientine, N,N′-diethyldithiocarbamate (DDC), 2,3,2′-tetraamine (2,3,2′-tet), neocuproine, N,N,N′,N′-tetrakis(2-pyridylmethyl)ethylenediamine (TPEN), 1,10-phenanthroline (PHE), tetraethylenepentamine, triethylenetetraamine and tris(2-carboxyethyl) phosphine (TCEP), ferrioxamine, CP94, EDTA, deferoxainine B (DFO) as the methanesulfonate salt (also known as desferrioxanilne B mesylate (DFOM)), desferal from Novartis (previously Ciba-Giegy), and apoferritin.

As used herein, a pharmaceutical composition is “stable” when the composition preserves the physical stability/integrity and/or chemical stability/integrity of the active pharmaceutical ingredient during storage. Furthermore, “stable pharmaceutical composition” is characterized by its level of degradation products not exceeding 5% at 40° C./75% RH after 6 months or 3% at 55° C./75% RH after two weeks, compared to their level in time zero.

As used herein, “effective” when referring to an amount of laquinimod refers to the quantity of laquinimod that is sufficient to yield a desired therapeutic response. Efficacy can be measured by e.g., a reduced intraocular pressure (IOP).

Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration. As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times weekly and so on, etc.

The route of administration can be, e.g., topical. Routes of administration can also be classified by whether the effect is local (e.g., in topical administration) or systemic (e.g., in enteral or parenteral administration). “Local administration” as used herein shall mean administration of a compound or composition directly to where its action is desired, and specifically excludes systemic administration. “Topical administration” of a compound or composition as used herein shall mean application of the compound or composition to body surfaces such as the skin or mucous membranes such as eyes. “Ocular administration” as used herein shall mean application of a compound or composition to the eye of a subject or to the skin around the eye (periocular skin) of a subject, i.e., local administration. Examples of ocular administration include topical administration directly to the eye, topical application to the eye lid or injection into a portion of the eye or eye socket. In addition, an “ocular pharmaceutical composition” as used herein means a pharmaceutical composition formulated for ocular administration.

“Treating” as used herein encompasses, e.g., inducing inhibition, regression, or stasis of a disease or disorder, e.g., glaucoma, or alleviating, lessening, suppressing, inhibiting, reducing the severity of, eliminating or substantially eliminating, or ameliorating a symptom of the disease or disorder.

“Inhibition” of disease progression or disease complication in a subject means preventing or reducing the disease progression and/or disease complication in the subject.

A “symptom” associated with glaucoma includes any clinical or laboratory manifestation associated with glaucoma and is not limited to what the subject can feel or observe.

As used herein, a subject “afflicted” with glaucoma means the subject has been diagnosed with glaucoma.

As used herein, a subject at “baseline” is as subject prior to administration of laquinimod in a therapy as described herein.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

It is understood that where a parameter range is provided, all integers within that range, and tenths thereof, are also provided by the invention. For example, “0.1-2.5 mg/day” includes 0.1 mg/day, 0.2 mg/day, 0.3 mg/day, etc. up to 2.5 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1: Assessment of the Neuroprotective Efficacy of Laquinimod for the Retinal Ganglion Cell (RGC) Survival in a Rat Glaucoma Model

The purpose of this study was to assess the efficacy of laquinimod in protecting against chronic ocular hypertension (OHT) and RGC degeneration in a rat model of glaucoma, created by injecting hypertonic saline into the episcleral veins in one eye of the Brown Norway rat. In this model, RGC degeneration occurs in response to increased IOP and OHT similar to that in human patients with glaucoma.

### SUMMARY

The study included 5 groups (n=8 each): Group 1 (1% Laquinimod for topical administration), Group 2 (Vehicle for topical administration), Group 3 (4% Laquinimod for topical administration), Group 4 (0.25% Laquinimod for topical administration) and Group 5 (0.1% Laquinimod for oral administration).

Prior to any experimental procedure, clinical observations were performed daily, body weights were obtained, intraocular pressure (IOP) was measured and detailed ocular examinations were performed on both eyes. Animals that were found to be clinically normal with no baseline ocular abnormalities were selected for use in the study.

The rat model of chronic ocular hypertension (OHT)/glaucoma was created in the left eye of each animal via two hypertonic saline injections (HSI) each one week apart. The un-injected right eye served as the control. Throughout the study, the vehicle control and the test articles were prepared weekly.

Rats were dosed once daily for oral administration (Group 5) and twice daily for topical administration groups (Groups 1-4), starting on the day of the first HSI until euthanasia. Detailed ocular examinations were performed one week after the 2nd HSI and on the day of euthanasia. Approximately one week prior to euthanasia, RGCs were retrogradely-labeled by bilateral injections of Fluoro-Gold (FG) into the superior colliculus in the brain. Post-dose IOP was measured weekly for 5 weeks starting one week after the 2nd HSI until euthanasia.

For each IOP measurement time-point following the 2nd HSI, the IOP elevation was calculated as the difference between the IOP in the left eye with OHT and that in the non-OHT right eye (ΔIOP). The ΔIOP for the 5 weekly post-HSI IOP measurements were averaged and constituted the Mean ΔIOP for each animal. Rats which did not have individual IOP measurements of 50 mmHg in the OHT eyes were selected from a larger pool and groups (n=8 each) were matched for the Mean ΔIOP. Other animals were removed from the study.

Animals were euthanized 5 weeks after the 2nd HSI. The retinas were flat-mounted, slide IDs were masked and 8 regions per retina were imaged using a confocal microscope. RGC at these regions were counted using the Image J software. After the optic nerves were extracted, they were plastic-embedded, sectioned and stained with Toluidine Blue at Schepens Eye Research Institute, Boston, Mass. The injury in the optic nerves was evaluated and graded by microscopic examination in a masked fashion.

There were no abnormal clinical signs or ocular abnormalities noted in any of the 40 animals in the beginning of the study. There were no treatment-related findings in the clinical observations or ocular examinations during the study in any of the animals.

A One-Way ANOVA indicated that the group body weights were not statistically different on the day of dosing among groups (P=0.957). Groups also did not differ in the amount of weight gained between the day of dosing and the day of euthanasia (One-Way ANOVA; P=0.559). For five weeks post-dose, the mean ΔIOP values (OHT−Non-OHT) (Mean±SD) were 9.1±2.2 mmHg, 9.1±2.6 mmHg, 9.3±2.7 mmHg, 9.2±1.5 mmHg, and 9.1±2.9 mmHg for Groups 1, 2, 3, 4 and 5, respectively. A One-Way ANOVA analysis indicated that the groups were well-matched for Mean ΔIOP (P>0.9). Compared to the non-OHT retinas, % Fluoro-gold labeled-RGC Loss (Mean±SD) in the OHT retinas was 17.6±33.8, 34.8±42.7, 26.8±36.4, 21.6±21.7, and 22.9±33.1 for Groups 1, 2, 3, 4 and 5, respectively. FG-Labeled RGC counts per mm² were compared between the Non-OHT and the OHT retinas for each group using two-tailed paired-t tests. Group 2 (vehicle topical; P=0.047) and Group 4 (0.25% Laquinimod topical; P=0.033) had fewer RGCs in the OHT eye compared to the Non-OHT eyes. The number of RGCs in Group 3 (4% Laquinimod topical; P=0.071) and Group 5 (0.1% Laquinimod oral; P=0.075) were statistically different between the OHT and Non-OHT eyes. However, in Group 1 (1% Laquinimod topical), there was no statistically significant difference in the RGC counts between the OHT and Non-OHT eyes (P=0.189). This suggests that the daily topical application of 1% Laquinimod may be neuroprotective for the RGC. For the optic nerves in the Non-OHT (control) eyes, the Mean Injury Grades (Mean±SD) were 1.1±0.1, 1.2±0.2, 1.2±0.2, 1.2±0.3 and 1.2±0.2 for Groups 1, 2, 3, 4 and 5, respectively. For the optic nerves in the OHT eyes, the Mean Injury Grades (Mean±SD) for the OHT optic nerves were 2.5±1.3, 3.0±1.6, 3.0±1.5, 2.8±1.3 and 2.7±1.5 for Groups 1, 2, 3, 4, and 5 respectively. As a secondary analysis, Mean ON Injury Grades were compared between the Non-OHT and the OHT optic nerves for each group using two-tailed paired-t tests. The Mean ON injury grades were significantly greater in the OHT eyes compared to the Non-OHT control eyes in all groups (P<0.05).

Materials

Test Article: laquinimod sodium stored at room temperature protected from light.

Control Article: 0% laquinimod sodium stored under refrigeration.

Animals

Number and Species: The study quote and outline-specified data were collected on 40 Brown Norway rats (Rattus norvegicus). Rats have been used historically as OHT models and there are no other approved alternative (non-animal methods). The study started with 90 animals to ensure that sufficient data were available at the end of the study.

Sex: male

Weight/Age Range: approximately 271.2-366.4 grams/at least 12 weeks old (adult) weighed to nearest 0.1 g.

Procedure

Ocular and oral exposure corresponds to the route of human exposure. For the four topical administration groups, the test and control articles were applied topically to the surface of one eye of the test system. The test article was administered orally for the fifth group of animals.

Preparation of Test and Control Articles

All test and control articles were prepared as described below. The final volume for the formulations described below is 200 mL but the formulations were proportionally changed to the required volume for each preparation. Three different concentrations (0.25%, 1%, and 4%) of the test article for topical application and the test article for oral gavage (0.1%) were prepared weekly. The control article for topical application was also prepared weekly. The identification of the test and control articles for the topical administration was masked after preparation of the topical formulations.

TABLE 1
LAQ 4.0% Topical Formulation (40 mg/mL of LAQ)

| Material | g/200 mL solution |
|---|---|
| Laquinimod Sodium | 8.52 (equivalent to 8 g |
| | Laquinimod acid) |
| Sodium Phosphate Dibasic 7 Hydrate | 2.497 |
| Sodium Phosphate Monobasic Monohydrate | 0.0942 |
| Hydroxy Ethyl Cellulose (HEC) HX | 0.3 |
| Water for Injection | Amount required to bring |
| | solution to 200 mL |

Table 1 Process:
1. The entire process is performed under yellow light or at dark conditions.
2. Weigh 175 g (175 mL) of water for injection in a glass container containing a stirrer.
3. Weigh and add the Sodium Phosphate Monobasic Monohydrate and Sodium Phosphate Dibasic 7 Hydrate and stir for approximately 5 minutes. Verify complete dissolution by visual inspection of a clear transparent solution.
4. Measure the pH (Approximately 7.7-8.3).
5. Weigh and add Laquinimod Sodium to the solution. Rinse the weighing boat from the Laquinimod Sodium remaining with ˜5 mL of water and to the solution. Stir for approximately 5 minutes and verify complete dissolution by visual inspection of a clear transparent solution.
6. Weigh and add the Hydroxy Ethyl Cellulose HX.
7. Stir for approximately 2 hours.
8. Remove the stirrer and add the solution to a 200 mL volumetric flask.
9. Add water for injection up to 200 mL.
10. Shake manually for approximately two minutes.
11. Transfer into an appropriate container. Wrap with aluminum foil and keep the solution refrigerated.

TABLE 2
LAQ 1.0% Topical Formulation (10 mg/mL of LAQ)

| Material | g/200 mL solution |
|---|---|
| Laquinimod Sodium | 2.12 (equivalent to 2 g |
| | Laquinimod acid) |
| Sodium Phosphate Dibasic 7 Hydrate | 2.497 |
| Sodium Phosphate Monobasic Monohydrate | 0.0942 |
| Hydroxy Ethyl Cellulose (HEC) HX | 0.3 |
| Sodium chloride | 0.75 |
| Water for Injection | Amount required to bring |
| | solution to 200 mL |

Table 2 Process:
1. The entire process is performed under yellow light or at dark conditions.
2. Weigh 175 g (175 mL) of water for injection in a glass container containing a stirrer.
3. Weigh and add the Sodium Phosphate Monobasic Monohydrate and Sodium Phosphate Dibasic 7 Hydrate and stir for approximately 5 minutes. Verify complete dissolution by visual inspection of a clear transparent solution.
4. Measure the pH (Approximately 7.7 to 8.3).
5. Weigh and add Laquinimod Sodium to the solution. Rinse the weighing boat from the Laquinimod Sodium remaining with ˜5 mL of water and to the solution. Stir for approximately 5 minutes and verify complete dissolution by visual inspection of a clear transparent solution.
6. Weigh and add the Sodium Chloride to the solution. Stir for approximately 2 minutes. Verify complete dissolution by visual inspection of a clear transparent solution.
7. Weigh and add the Hydroxy Ethyl Cellulose HX.
8. Stir for approximately 2 hours.
9. Remove the stirrer and add the solution to a 200 mL volumetric flask.
10. Add water for injection up to 200 mL.
11. Shake manually for approximately two minutes.
12. Transfer into an appropriate container. Wrap with aluminum foil and keep the solution refrigerated.

TABLE 3
LAQ 0.25% Topical Formulation (2.5 mg/mL of LAQ)

| Material | g/200 mL solution |
|---|---|
| Laquinimod Sodium | 0.54 (equivalent to 0.5 g |
| | Laquinimod acid) |
| Sodium Phosphate Dibasic 7 Hydrate | 2.497 |
| Sodium Phosphate Monobasic Monohydrate | 0.0942 |
| Hydroxy Ethyl Cellulose (HEC) HX | 0.3 |
| Sodium chloride | 0.94 |
| Water for Injection | Amount required to bring |
| | solution to 200 mL |

Table 3 Process:
1. The entire process is performed under yellow light or at dark conditions.
2. Weigh 175 g (175 mL) of water for injection in a glass container containing a stirrer.
3. Weigh and add the Sodium Phosphate Monobasic Monohydrate and Sodium Phosphate Dibasic 7 Hydrate and stir for approximately 5 minutes. Verify complete dissolution by visual inspection of a clear transparent solution.
4. Measure the pH (Approximately 7.7-8.3).
5. Weigh and add Laquinimod Sodium to the solution. Rinse the weighing boat from the Laquinimod Sodium remaining with ˜5 mL of water and to the solution. Stir for approximately 5 minutes and verify complete dissolution by visual inspection of a clear transparent solution.
6. Weigh and add the Sodium Chloride to the solution. Stir for approximately 2 minutes. Verify complete dissolution by visual inspection of a clear transparent solution.
7. Weigh and add the Hydroxy Ethyl Cellulose HX.
8. Stir for approximately 2 hours.
9. Remove the stirrer and add the solution to a 200 mL volumetric flask.
10. Add water for injection up to 200 mL.
11. Shake manually for approximately two minutes.
12. Transfer into an appropriate container. Wrap with aluminum foil and keep the solution refrigerated.

TABLE 4
LAQ 0% Topical Formulation (Control Article)

| Material | g/200 mL solution |
|---|---|
| Sodium Phosphate Dibasic 7 Hydrate | 2.497 |
| Sodium Phosphate Monobasic Monohydrate | 0.0942 |
| Hydroxy Ethyl Cellulose (HEC) HX | 0.3 |
| Sodium chloride | 1 |
| Water for Injection | Amount required to bring |
| | solution to 200 mL |

Table 4 Process:
1. The entire process is performed under yellow light or at dark conditions.
2. Weigh 175 g (175 mL) of water for injection in a glass container containing a stirrer.
3. Weigh and add the Sodium Phosphate Monobasic Monohydrate and Sodium Phosphate Dibasic 7 Hydrate and stir for approximately 5 minutes. Verify complete dissolution by visual inspection of a clear transparent solution.
4. Measure the pH (Approximately 7.7 to 8.3).
5. Weigh and add the Sodium Chloride to the solution. Stir for approximately 2 minutes. Verify complete dissolution by visual inspection of a clear transparent solution.
6. Weigh and add the Hydroxy Ethyl Cellulose HX.
7. Stir for approximately 2 hours.
8. Remove the stirrer and add the solution to a 200 mL volumetric flask.
9. Add water for injection up to 200 mL.
10. Shake manually for approximately two minutes.
11. Transfer into an appropriate container.

TABLE 5
LAQ 0.1% Oral Formulation (1.0 mg/mL of LAQ)

| Material | g/200 mL solution |
|---|---|
| Laquinimod Sodium | 0.2 (Equivalent to 0.2 g laquinimod acid) |
| Autoclaved Tap Water | Amount required to bring solution to 200 mL |

Table 5 Process:
1. The entire process is performed under yellow light or at dark conditions.
2. Weigh 150 g (150 mL) of autoclaved tap water in a glass container containing a stirrer.
3. Weigh and add Laquinimod Sodium to the solution. Rinse the remaining Laquinimod Sodium in the weighing boat with ˜5 mL of water and add to the solution. Stir for approximately 5 minutes and verify complete dissolution by visual inspection of a clear transparent solution.
4. Add autoclaved tap water up to 200 mL.
5. Stir for 5 minutes.
6. Transfer into an appropriate amber

Pre-Dose Administration and Selection of Animals:

Clinical observations were performed daily according to Table 6 below:

TABLE 6
Clinical Signs and Observations

| | Observed Sign | Involved System(s) |
|---|---|---|
| Respiratory | Dyspnea (abdominal breathing, | CNS, pulmonary, cardiac |
| | gasping), apnea, cyanosis, | |
| | tachypnea, nostril discharges | |
| Motor Activities | Decrease/increase somnolence, | CNS, somatomotor, sensory, |
| | loss of righting, anaesthesia, | neuromuscular, autonomic |
| | catalepsy, ataxia, unusual | |
| | locomotion, prostration, tremors, | |
| | fasciculation | |
| Convulsion | Clonic, tonic, tonic-clonic, | CNS, neuromuscular, autonomic, |
| | asphyxial, opisthotonos | respiratory |
| Reflexes | Corneal, righting, myotact, light, | CNS, sensory, autonomic, |
| | startle reflex | neuromuscular |
| Ocular Signs | Lacrimation, miosis, mydriasis, | Autonomic, irritation |
| | exophthalmos, ptosis, opacity, | |
| | iritis, conjunctivitis, | |
| | chromodacryorrhea, relaxation of | |
| | nictitating membrane | |
| Cardiovascular Signs | Bradycardia, tachycardia, | CNS, autonomic, cardiac, |
| | arrhythmia, vasodilation, | pulmonary |
| | vasoconstriction | |
| Salivation | Excessive | Autonomic |
| Piloerection | Rough hair | Autonomic |
| Analgesia | Decrease reaction | CNS, sensory |
| Muscle Tone | Hypotonia, hypertonia | Autonomic |
| Gastrointestinal | Soft stool, diarrhea, emesis, | CNS, autonomic, sensory, GI |
| | diuresis, erythruria | motility, kidney |
| Skin | Edema, erythema | Tissue damage, irritation |

Animals were weighed weekly prior to initiation of dosing. The first day the rats are weighed was designated as Study Day 1, and the beginning of Study Week 1.

Ophthalmic Examinations:

Animals selected for the study were examined prior to the initial administration of the test or the control articles to ensure that both eyes were free of abnormality, damage, and disease. Both eyes were examined, scored and recorded prior to the initial dose administration using a hand held slit-lamp and a direct ophthalmoscope or the surgical microscope according to the Classification System for Grading Ocular Lesions as described in Table 7, and the Ocular Posterior Segment Scoring Scale of Table 8. Posterior segment examination was performed after topical application of Tropicamide in the conscious state, or after the rats are anesthetized with isoflorane inhalation. Only rats showing no signs of eye irritation, ocular defects, or preexisting corneal injury were used in the study.

TABLE 7
Combined Draize and McDonald-Shadduck Scoring System
CONJUNCTIVA

| | A. REDNESS/CONGESTION |
|---|---|
| 0 | Vessels normal. |
| | May appear blanched to reddish pink without perilimbal injection (except at 12:00 and 6:00 |
| | o'clock positions) with vessels of the palpebral and bulbar conjunctivae easily observed. |
| 1 | Vessels definitely injected above normal. |
| | A flushed, reddish color predominately confined to the palpebral conjunctivae with some |
| | perilimbal injection but primarily confined to the lower and upper parts of the eye from, the 4:00 |
| | and 7:00 and 11:00 to 1:00 o'clock positions. |
| 2*† | More diffuse, deeper crimson red, individual vessels not easily discernible. |
| | Bright red color of the palpebral conjunctiva with accompanying perilimbar injection covering |
| | at least 75% of the circumference of the perilimbal region. |
| 3*† | Diffuse beefy red. |
| | Dark beefy red color with congestion of both the bulbar and palpebral conjunctiva along with |
| | pronounced perilimbal injection and the presence of petechia on the conjunctiva. The |
| | B. CHEMOSIS: |
| 0 | Normal. No swelling of the conjunctival tissue. |
| 1 | Any swelling above normal (includes nictitating membrane). |
| | Swelling above normal without eversion of the lids (can be easily ascertained by noting that the |
| | upper and lower eyelids are positioned as in the normal eye); swelling generally starts in the |
| | lower cul-de-sac near the inner canthus which needs slit-lamp examination. |
| 2*† | Obvious swelling with partial eversion of lids. |
| | Swelling with misalignment of the normal approximation of the lower and upper eyelids; |
| | primarily confined to the upper eyelid so that in the initial stages the misapproximation of the |
| | eyelids begins by partial eversion of the upper eyelid. In this stage, swelling is confined |
| | generally to the upper eyelid, although it exists in the lower cul-de-sac. |
| | Swelling with lids about half closed. |
| 3*† | Swelling definite with partial eversion of the upper and lower eyelids essentially equivalent. |
| | This can be easily ascertained by looking at the animal straight head on and noticing the |
| | positioning of the eyelids; if the eye margins do not meet, eversion has occurred. |
| 4*† | Swelling with lids about half closed to completely closed. |
| | Eversion of the upper eyelid is pronounced with less pronounced eversion of the lower eyelid. It |
| | is difficult to retract the lids and observe the perilimbal region. |
| | C. DISCHARGE: |
| 0 | Normal or no discharge. |
| 1 | Any amount different from normal (does not include small amounts observed in inner canthus of |
| | normal animals). |
| | Discharge above normal and present on the inner portion of the eye but not on the lids or hairs |
| | of the eyelids. One can ignore the small amount that is in the inner and outer canthus if it has not |
| | been removed prior to starting the study. |
| 2 | Discharge with moistening of lids and hairs just adjacent to lids. Discharge is abundant, easily |
| | observed, and has collected on the lids around the hairs of the eyelids. |
| 3 | Discharge with moistening of the lids and hairs, and considerable area around the eye. |

CORNEA

| | |
|---|---|
| | D. OPACITY-degree of density (areas most dense taken for reading): |
| 0 | No ulceration or opacity. |
| | Normal cornea. Appears with the slit lamp as having a bright gray line on the endothelial surface |
| | and a bright gray line on the endothelial surface with a marble-like gray appearance of the |
| | stroma. |
| 1* | Scattered or diffuse areas of opacity (other than slight dulling of normal luster), details of iris |
| | clearly visible. Some loss of transparency. Only the anterior one-half of the stroma is involved |
| | as observed with an optical section of the slit lamp. The underlying structures are clearly visible |
| | with diffuse illumination, although some cloudiness can be readily apparent with diffuse |
| | illumination. |
| 2*† | Easily discernible translucent areas, details of iris slightly obscured. Moderate loss of |
| | transparency. In addition to involving the anterior stroma, the cloudiness extends all the way to |
| | the endothelium. The stroma has lost its marble-like appearance and is homogeneously white. |
| | With diffuse illumination, underlying structures are clearly visible. |
| 3*† | Opalescent/nacreous areas, no details of iris visible, size of pupil barely discernible. |
| | Involvement of the entire thickness of the stroma. With optical section, the endothelial surface is |
| | still visible. However, with diffuse illumination the underlying structures are just barely visible |
| | (to the extent that the observer is still able to grade flare, iritis, observe for pupillary response, |
| | and note lenticular changes. |
| 4*† | Opaque cornea, iris not discernible through opacity. |
| | Involvement of the entire thickness of the stroma. With the optical section, cannot clearly |
| | visualize the endothelium. With diffuse illumination, the underlying structures cannot be seen. |
| | Cloudiness removes the capability of judging and grading aqueous flare, iritis, lenticular |
| | changes, and pupillary response. |
| | E. AREAS OF CORNEA INVOLVED: |
| 0 | Normal cornea with no area of cloudiness. |
| 1 | One-quarter (or less), but not zero. |
| 2 | Greater than one-quarter, but less than one-half. |
| 3* | Greater than one-half, but less than three-quarters. |
| 4* | Greater than three-quarters, up to whole area. |
| | F. FLUORESCEIN STAINING: |
| 0 | Absence of fluorescein staining. |
| 1 | Slight fluorescein staining confined to a small focus. With diffuse illumination the underlying |
| | structures are easily visible. The outline of the papillary margin is as if there were no fluorescein |
| | staining. |
| 2 | Moderate fluorescein staining confined to a small focus. With diffuse illumination the |
| | underlying structures are clearly visible, although there is some loss of detail. |
| 3 | Marked fluorescein staining. Staining may involve a larger portion of the cornea. With diffuse |
| | illumination the underlying structures are barely visible but are not completely obliterated. |
| 4 | Extreme fluorescein staining. With diffuse illumination the underlying structures cannot be |
| | observed. |
| | G. CORNEA PANNUS: |
| 0 | No pannus |
| 1 | Vascularization is present but vessels have not invaded the entire corneal circumference. Where |
| | localized vessel invasion has occurred, they have not penetrated beyond 2 mm. |
| 2 | Vessels have invaded 2 mm or more around the entire corneal circumference. |

IRIS

| | |
|---|---|
| 0 | Normal iris without any hyperemia of the iris vessels. Occasionally around the 12:00 to 1:00 |
| | position near the pupillary border and the 6:00 and 7:00 position near the pupillary border there |
| | is a small area around 1-3 mm in diameter in which both the secondary and tertiary vessels are |
| | slightly hyperemic. |
| 1*† | Folds above normal, congestion, swelling, circumcorneal injection (any or all of these or |
| | combination of any thereof), iris still reacting to light (sluggish reaction is positive). Minimal |
| | injection of secondary vessels but not tertiary. Generally, it is uniform, but may be of greater |
| | intensity at the 1:00 or 6:00 position, the tertiary vessels must be substantially hyperemic. |
| 2*† | No reaction to light, hemorrhage, gross destruction (any or all of these). Minimal injection of |
| | tertiary vessels and minimal to moderate injection of the secondary vessels. |
| 3*† | Moderate injection of the secondary and tertiary vessels with slight swelling of the iris stroma |
| | (this gives the iris surface a slightly rupose appearance which is usually most prominent near the |
| | 3:00 and 9:00 positions). |
| 4*† | Marked injection of the secondary and tertiary vessels with marked swelling of the iris stroma. |
| | The iris appears rugose; may be accompanied by hemorrhage (hyperemia) in the anterior |
| | chamber. |

AQUEOUS FLARE

| | |
|---|---|
| 0 | Absence of visible light beam in the anterior chamber (no Tyndall effect). |
| 1 | Tyndall effect is barely discernible. The intensity of the light beam in the anterior chamber is |
| | less than the density of the slit beam as it passes through the lens. |
| 2 | The Tyndall effect in the anterior chamber is easily discernible and is of equal intensity as the |
| | density of the slit beam as it passes through the lens. |
| 3 | The Tyndall effect in the anterior chamber is easily discernible; its intensity is greater than the |
| | intensity of the slit beam as it passes through the lens. |

PUPILLARY LIGHT REFLEX:

| | |
|---|---|
| 0 | Normal pupillary light reflex |
| 1 | Sluggish pupillary light reflex |
| 2 | No pupillary light reflex |

LENS

| | |
|---|---|
| 0 | Normal |
| 1 | The presence of lenticular opacities should be described and the location noted as defined |
| | below: |
| | Anterior capsule |
| | Anterior subcapsule |
| | Anterior cortical |
| | Nuclear |
| | Posterior cortical |
| | Posterior subcapsule |
| | Posterior capsule |

*= Positive Reaction (ISO)
†= Positive Reaction (OECD)

TABLE 8
Ocular Posterior Segment Scoring Scale
VITREOUS BODY

| | |
|---|---|
| 0 | Normal, the vitreous body is clear or transparent. |
| 1 | Abnormal, the vitreous body is not clear or not transparent and homogenous gel that fills the |
| | space between the posterior axial lens capsule, posterior chamber, and ocular fundus. |

OPTIC DISC/OPTIC NERVE

| | |
|---|---|
| 0 | Normal, the optic disc and optic nerve are with light red color, cupping size normal (cup-to-disc |
| | ratio <0.2), and normal sharpness of edge, without swelling, hemorrhages, notching in the optic |
| | disc and any other unusual anomalies. |
| 1 | Abnormal, the optic disc and optic nerve are not with light red color or cupping size normal |
| | (cup-to-disc ratio >0.2), and no normal sharpness of edge or with swelling, hemorrhages, |
| | notching in the optic disc and any other unusual anomalies. |

RETINAL BLOOD VASCULATURE

| | |
|---|---|
| 0 | Normal, the retinal arteries and veins fill in blood and normalize sharpness without hemorrhage |
| | and exudation. |
| 1 | Abnormal, the retinal arteries and veins don't fill in blood and don't have normalize sharpness or |
| | with hemorrhage and exudation. |

RETINAL HEMORHAGE, EXUDATION, AND DETACHMENT

| | |
|---|---|
| 0 | Normal retina with no area of hemorrhage or/and exudation or/and detachment |
| 1 | Retinal hemorrhage or/and exudation or/and detachment ≤1 quadrant area |
| 2 | 1 quadrant area < retinal hemorrhage or/and exudation or/and detachment ≤ 2 quadrant areas |
| 3 | 2 quadrant areas < hemorrhage or/and exudation or/and detachment ≤ 3 quadrant areas |
| 4 | 3 quadrant areas < retinal hemorrhage or/and exudation or/and detachment exudation ≤ 4 |
| | quadrant areas |

CHOROIDAL HEMORRHAGE, EXUDATION, AND DETACHMENT

| | |
|---|---|
| 0 | Normal choroid with no area of choroidal and hemorrhage or/and exudation or/and detachment |
| 1 | Choroidal hemorrhage or/and exudation or/and detachment |
| | ≤1 quadrant area |
| 2 | quadrant area < Choroidal hemorrhage or/and exudation or/and detachment ≤ 2 quadrant areas |
| 3 | 2 quadrant areas < Choroidal hemorrhage or/and exudation or/and detachment ≤ 3 quadrant |
| | areas |
| 4 | quadrant areas < Choroidal hemorrhage or/and exudation or/and detachment ≤ 4 quadrant area |

Intraocular Pressure (IOP) Measurements:

Baseline IOP measurements were taken before the initial dose administration. After application of topical anesthesia (0.5% Proparacaine HCl Ophthalmic Solution), IOP was measured on conscious rats on both eyes using a Tono-Pen Vet tonometer (Reichert, Inc.; Depew, N.Y.). Ten (10) IOP readings are recorded from each eye and averaged. IOP measurements were taken around the same time (e.g., between 10 a.m. and 2 p.m.) across measurement time-points to minimize the circadian variability of IOP.

Dose Administration:

Rats were separated in 5 groups. Animals in each group receive one of the five following articles during the study:
1. Control vehicletopical
2. 0.25% Laquinimod topical
3. 1% Laquinimod topical
4. 4% Laquinimod topical
5. 0.1% Laquinimod oral

Topical Dosing:

Rats were dosed topically only on the surface of the left eye in which OHT was induced. No article was administered on the un-operated right eye which served as the control.

The topical dose was administered on the surface of the left eye using a calibrated micro-pipette and a sterile tip. The volume for each topical dose was 10 μL.

Rats were dosed twice daily, starting on the day of the first HSI until euthanasia. The first daily dose was administered approximately between 8 a.m. and 9 a.m. The second daily dose was administered approximately between 4 p.m. and 5 p.m. On the day of euthanasia, rats were dosed only once in the morning approximately between 8 a.m. and 9 a.m.

Oral Dosing:

Rats were dosed orally daily approximately between 8 a.m. and 10 a.m., starting on the day of the first HSI until euthanasia. The last day of dosing was the day of euthanasia. The volume for each oral dose was 1 mL.

Post-Dose Procedures:

Chronic ocular hypertension (OHT) was created through two hypertonic saline injections (HSI) which were performed one week apart in the left eye. For each HSI, after the rats were anesthetized with a suitable anesthetic, a suture thread was passed through the left eyelid to fix it open. A local anesthetic (e.g., 0.5% Proparacaine HCl Ophthalmic Solution) was applied on the surgery eye topically. The conjunctiva was incised with Vannas scissors to expose an episcleral vein. An occluder ring with a groove was fitted around the left eye to provide unobstructed passage for the selected episcleral vein while obstructing the other episcleral veins. Using a pulled-glass needle, 50-250 μL of 1.8M hypertonic saline solution was injected into the exposed episcleral vein to scar the aqueous humor outflow pathway in an attempt to elevate IOP. The ring was removed shortly after the injection. The un-operated right eye served as the control. Ophthalmic ointment was applied to both eyes to prevent corneal damage. For each HSI procedure, buprenorphine was administered subcutaneously for approximately 24 hours to manage post-operative pains as appropriate. Clinical observations was performed at least once daily as described in Table 6.

Moribund and Dead Animals:

Animals were observed once daily for moribundity/mortality as part of the clinical observations. There were no moribound animals.

Measurements and Criterion:

IOP measurements were taken once weekly starting one week after the second HSI until euthanasia (a total of 5 measurements). After application of topical anesthesia (0.5% Proparacaine HCl Ophthalmic Solution), IOP was measured on conscious rats on both eyes using a Tono-Pen Vet tonometer (Reichert, Inc.; Depew, N.Y.). For each time-point, ten (10) IOP readings were recorded from each eye and averaged. IOP measurements were taken around the same time (e.g., between 10 a.m. and 2 p.m.) across measurement time-points to minimize the circadian variability of IOP.

The IOP measurements were evaluated as follows: For each time-point following the HSI, the IOP elevation was calculated as the difference between the level in the left eye with OHT and that in the normal right eye (ΔIOP). The ΔIOP of the 5-weekly post-HSI IOP measurements were averaged and constitute the Mean ΔIOP for each animal. For each group, ten (10) animals with a sustained IOP elevation in the OHT eye were selected from a larger pool and groups are matched for the Mean ΔIOP. Other animals were removed from the study and euthanized. Data was analyzed and reported for rats which did not have individual IOP measurements of 50 mmHg in the OHT eyes.

Ophthalmic Examinations:

Both eyes were examined and scored at one week after the second HSI and on the day of euthanasia, for a total of two times. Ophthalmic examinations were performed according to the Classification System for Grading Ocular Lesions as described in Table 7, and the Ocular Posterior Segment Scoring Scale of Table 8 using a hand-held slit-lamp and a direct ophthalmoscope or the surgical microscope. Posterior segment examination was performed after the topical application of Tropicamide in the conscious state, or after the rats are anesthetized with isoflorane inhalation.

Fluoro-Gold (FG) Back-Labeling of Retinal Ganglion Cells (RGC):

Approximately one week prior to euthanasia, RGC was labeled with the retrograde tracer FG. First, animals were sedated with appropriate anesthesia. Using a stereotaxic device, RGC was back-labeled with an injection of 2.5 μL of 4% FG into the superior colliculus in each hemisphere. Rats received subcutaneous injections of buprenorphine for approximately 48 hours to manage post-surgery pain as appropriate.

Animals were euthanized by CO₂ inhalation 5-weeks after the 2′^{d} HSI.

Extraction and Processing of the Eyes and the Optic Nerves (ON):

The eyes were immediately enucleated with the optic nerve attached. An approximately 2.0 mm piece of the ON proximal to the globe is separated and labeled with tissue mark to indicate the orientation of the nerve. The ON piece is placed in Modified Karnovsky's Fixative in 0.1M Na cacodylate buffer and kept at 4±2° C. overnight. The next day ON piece was washed at least three times for at least 10 minutes each in 0.1M Na cacodylate buffer and stored in 0.1M Na cacodylate buffer at 4±2° C. After specimens were transferred to a contracted processing facility, within a week, the optic nerves were processed for plastic embedding: After post-fixation in 2% osmium tetroxide in 0.1M Na cacodylate buffer for at least 1.5 hours, ONs were dehydrated in graded ethanols, transitioned in propylene oxide and infiltrated with propylene oxide and epon mixtures. One to five (1-5) μm-thick cross-sections were taken via a microtome at the ON end approximately 2.0 mm away from the globe. ON sections were stained with 1% toluidine blue and cover-slipped. The processed specimens were returned. The identification of the ONs were masked prior to injury analysis. The ON cross-sections were analyzed for injury by light microscopy as follows:

The damaging effect of the sustained IOP elevation was assessed by qualitative microscopic analysis of the ON cross-sections using a well-established grading system described in Table 9. This method allowed damage analysis of the entire retinal ganglion cell output (the ON) in one section by light microscopy and was more sensitive than counting total axons especially if there is mild nerve damage. Sustained IOP elevation resulted in degenerating, swollen axons and collapsed myelin sheaths in the optic nerve. The extent of injury was then graded by light microscopy based on a pattern of damage observed in rats with elevated IOP.

TABLE 9
Grading Scale for Optic Nerve Injury

| | |
|---|---|
| Grade 1 (Normal) | Normal optic nerve with healthy axons. |
| Grade 2 (FOCAL INJURY) | Degenerating axons with myelin debris are stained densely |
| | and appear focally. Some axonal swellings are present. |
| Grade 3 (INJURY SPREADING AWAY | Several degenerating axons with myelin debris and axonal |
| FROM FOCAL) | swellings spread away from the focal area. Normal axons |
| | still predominate. |
| Grade 4 (WIDE-SPREAD INJURY; | Several degenerating axons with myelin debris and axonal |
| EQUIVALENT NUMBER of | swellings are present throughout the nerve. There are |
| DEGENERATING and NORMAL AXONS) | approximately equal numbers of normal and abnormal |
| | axons. |
| Grade 5 (WIDE-SPREAD INJURY; | Degenerating axons with myelin debris and swollen axons |
| DEGENERATING AXONS LARGELY | largely dominate the optic nerve, with gliosis in severe |
| DOMINATE) | cases. |

The eyes were fixed in 4% paraformaldehyde (PFA) fixative at 4±2° C. for at least 24 hours. Retinas were dissected and flat whole-mounted for confocal visualization and imaging. The actual identification of the retinal flat-mounts was masked prior to confocal imaging. Eight regions per retina were imaged using a confocal microscope and the RGC in each region were counted as follows:

Retinal images were evaluated by confocal fluorescence microscopic examination. A three-dimensional view of the x-axis, y-axis, and z-axis were designed and processed using a specific system of image analysis software (Leica Confocal Software) to obtain an image of the viable RGCs labeled with FG. Two areas which were approximately 1.5 mm and 2.75 mm away from the center of the ON head were selected in each retinal quadrant (8 regions per retina) and serial images of the retinal ganglion cell layer were taken by the Confocal Microscope. A two-dimensional maximum projection image of the serial images was used to count the viable RGCs using an image analysis software. The number of viable RGCs per image was expressed in mm².

Evaluation Criteria

The results of the study were considered in terms of the in-life observations and any microscopic observations.

IOP Criterion:

For each time-point following the HSI, the IOP elevation was calculated as the difference between the level in the left eye with OHT and that in the normal right eye (ΔIOP). The ΔIOP of the 5-weekly post-HSI IOP measurements were averaged and constitute the Mean ΔIOP for each animal. For each group, ten (10) animals with a sustained IOP elevation in the OHT eye were selected from a larger pool and groups were matched for the Mean ΔIOP. Other animals were removed from the study and euthanized. Data was analyzed and reported for rats which do not have individual IOP measurements of ≥50 mmHg in the OHT eyes.

Imaging of the Retinas by Confocal Microscopy:

Retinal images were evaluated by confocal fluorescence microscopic examination. A three-dimensional view of the x-axis, y-axis, and z-axis were designed and processed using a specific system of image analysis software (Leica Confocal Software) to obtain an image of the viable RGCs labeled with FG. Two areas which were approximately 1.5 mm and 2.75 mm away from the center of the ON head were selected in each retinal quadrant (8 regions per retina) and serial images of the retinal ganglion cell layer are taken by the Confocal Microscope. A two-dimensional maximum projection image of the serial images was used to count the viable RGCs using an image analysis software. The number of viable RGCs per image was expressed in mm².

Percent RGC loss in the OHT retinas was calculated in comparison to the RGC counts in the Non-OHT retina of the same animal using the following formula: (100−(100×OHT/Non-OHT Mean RGC Counts per Retina)). The RGC counts in each Non-OHT retina were considered 100% for that animal.

The damaging effect of the sustained IOP elevation was assessed by qualitative microscopic analysis of the ON cross-sections using a well-established grading system described in Table 9.

This method allowed damage analysis of the entire retinal ganglion cell output (the ON) in one section by light microscopy and was more sensitive than counting total axons especially if there is mild nerve damage. Sustained IOP elevation resulted in degenerating, swollen axons and collapsed myelin sheaths in the optic nerve. The extent of injury was then graded by light microscopy based on a pattern of damage observed in rats with elevated IOP.

Data Analysis:

Initially One-Way ANOVA was used to address statistically significant differences among groups. If there was a statistical significance, data of the test groups was further compared with the data of the control group using Dunnett's multiple comparison tests. Two-Way ANOVA, paired-t-tests, % neuroprotection calculations were also performed. Any differences between control and test animals was considered statistically significant only if the probability of the differences being due to chance is equal to or less than 5% (p≤0.05; two-tailed). Statistical analysis is performed using Minitab, Minitab Inc, State College, Pa. Any significant difference is further assessed for biological relevance by comparison to the literature and historical data.

Results

TABLE 10
% RGC Loss (100 − (100 × OHT/Non-OHT Mean RGC Counts per
Retina))

| | Route/ | | |
|---|---|---|---|
| | Laquinimod | Mean % RGC | |
| Group | Percentage | Loss | SD % RGC Loss |
| 1 | Topical/1% | 17.6 | 33.8 |
| 2 | Topical/ | 34.8 | 42.7 |
| | Vehicle | | |
| 3 | Topical/4% | 26.8 | 36.4 |
| 4 | Topical/0.25% | 21.6 | 21.7 |
| 5 | Oral/0.1% | 22.9 | 33.1 |

RGC = Retinal Ganglion Cell
OHT = Ocular Hypertension
SD = Standard Deviation

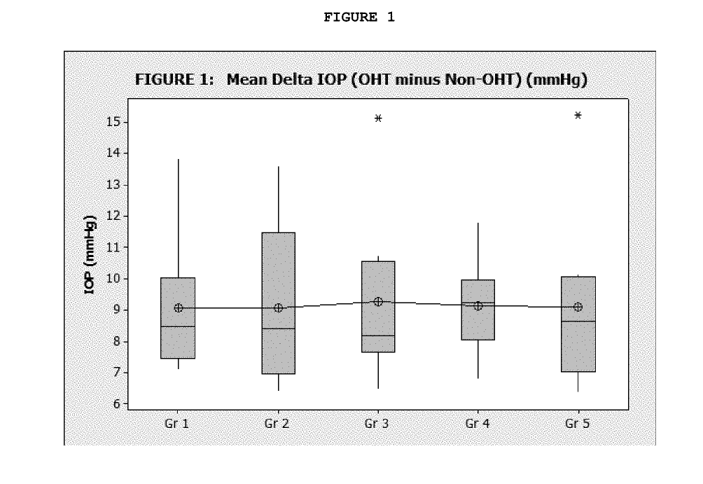

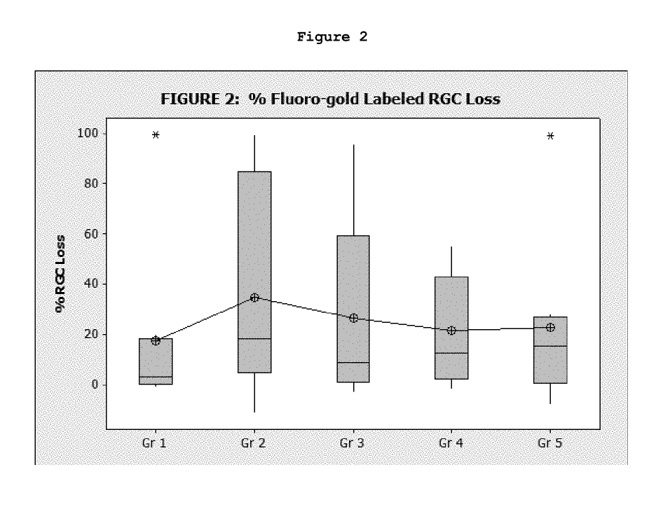

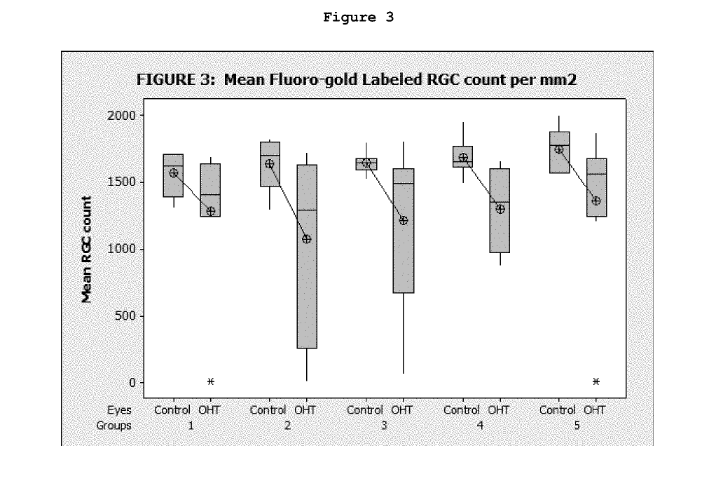

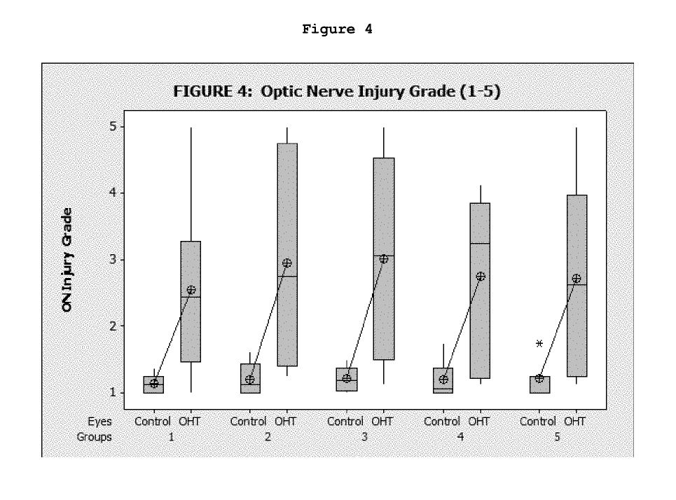

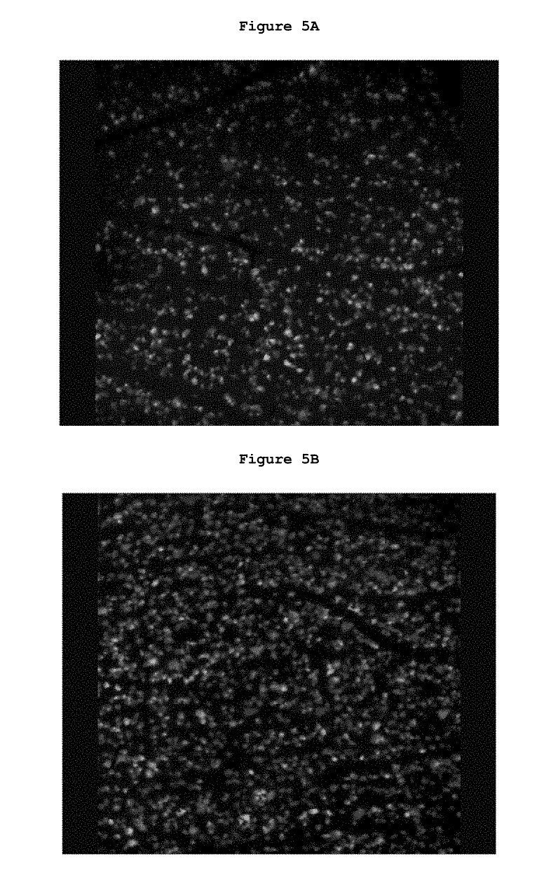

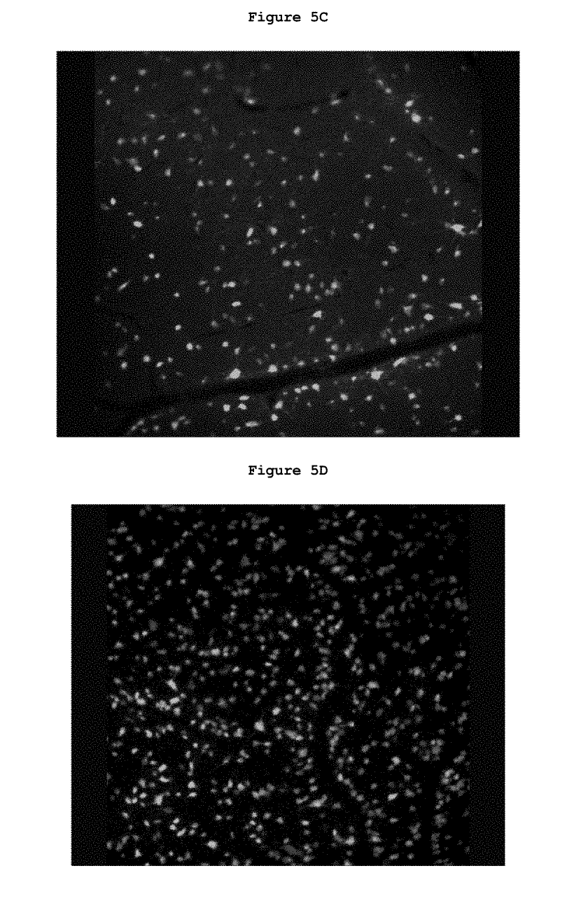

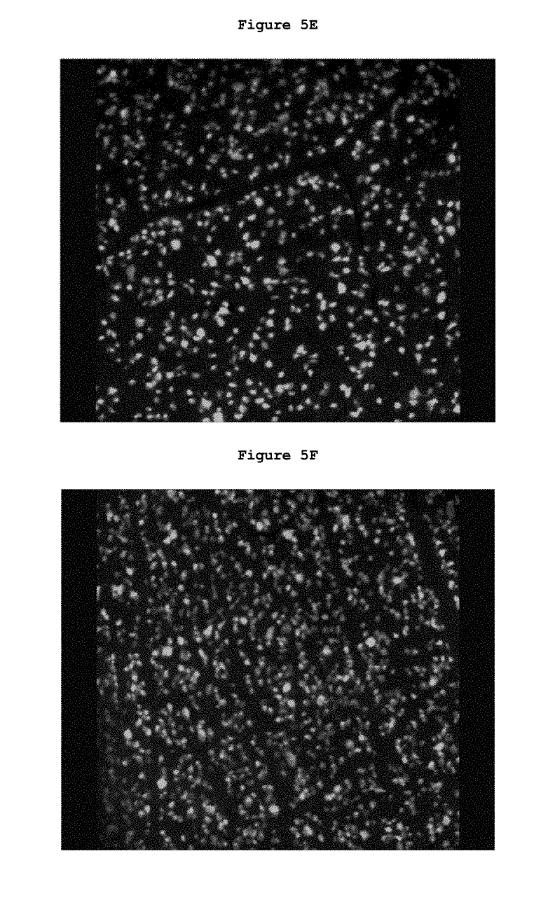

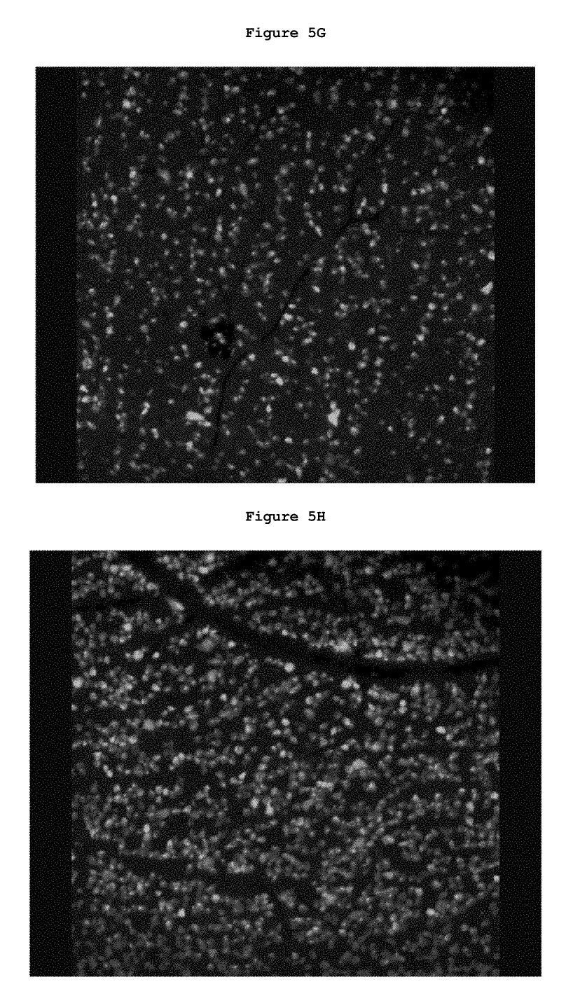

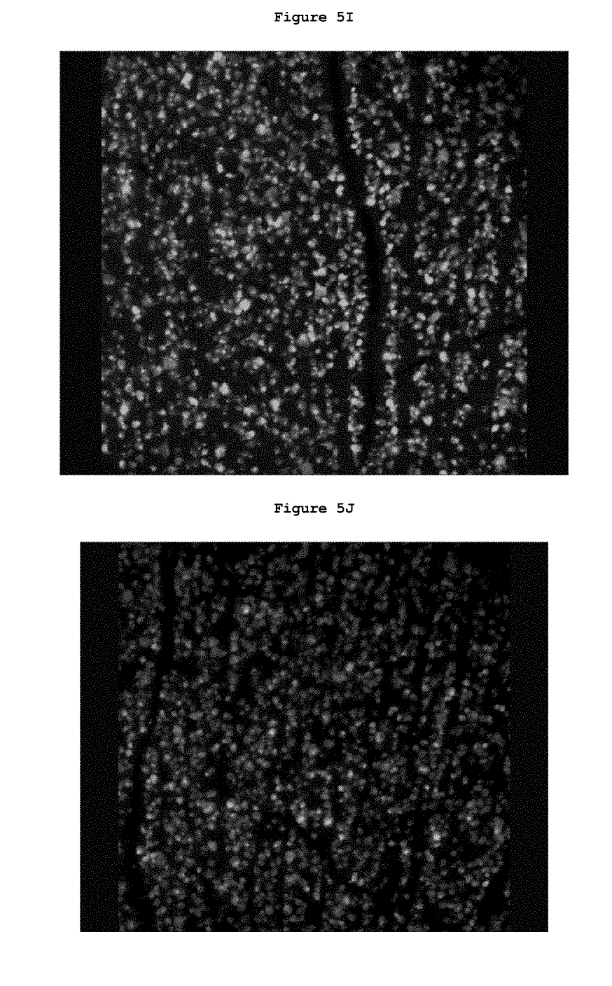

1. Per protocol, data were analyzed and reported for forty (40) rats which did not have individual IOP measurements of 50 mmHg in the OHT eyes.
2. Body Weights: The range of the baseline body weights of the 40 rats from Groups 1-5 was 258.6-351.3 grams at the start of the study. On the day of dosing and the first HSI, body weight range of all rats was 271.2-366.4 grams. A One-Way ANOVA indicated that the group body weights were not statistically different on the day of dosing (P=0.957). Rats weighed 271.4-349.3 grams on the day of euthanasia. Groups did not differ in the amount of weight gained between the day of dosing and the day of euthanasia (One-Way ANOVA; P=0.559).
3. Clinical Observations: There were no abnormal clinical observations in the beginning of the study at the time of animal assignment. Following the HSI surgeries, swelling of the surgery eye, swelling of the conjunctiva and scleral/corneal discoloration in the surgery eye were observed in all groups. Following the Fluoro-Gold brain injection surgeries, hair loss and skin wounds were noted in all groups due to the hair on the skull being shaved prior to surgery and the skin on the skull being incised and sutured after surgery. These observations were expected and related with the glaucoma model creation (HSI) and the Fluoro-gold brain injection surgeries. Abnormal clinical observations which were not related with the HSI and FG injection surgeries were hair loss in an animal from Group 2 and in an animal from Group 5, and skin wound in 2 animals from Group 5. Since Group 2 was the vehicle control group, these abnormalities were incidental and not test-article related.
4. IOP Measurements (mmHg): None of the animals included in the study had individual IOP measurements of ≥50 mmHg in the OHT eyes. The Mean ΔIOP (OHT−Non-OHT) (Mean±SD) was 9.1±2.2 mmHg, 9.1±2.6 mmHg, 9.3±2.7 mmHg, 9.2±1.5 mmHg, and 9.1±2.9 mmHg for Groups 1, 2, 3, 4 and 5, respectively. A One-Way ANOVA analysis indicated that the groups were well-matched for Mean ΔIOP (P>0.9) (FIG. 1). Representative images of the retinas with FG-labeled RGC are shown in FIGS. 5A-5J. % FG-Labeled RGC loss in the OHT retinas compared with the corresponding non-OHT retinas is shown in Table 10. Compared to the non-OHT retinas, % RGC Loss (Mean±SD) in the OHT retinas was 17.6±33.8, 34.8±42.7, 26.8±36.4, 21.6±21.7, and 22.9±33.1 for Groups 1, 2, 3, 4 and 5, respectively (FIG. 2).
5. FG-labeled RGC counts: A Two-Way ANOVA on the RGC counts in the OHT and Non-OHT (control) eyes versus groups did not show a significant eye versus group interaction (P=0.919), indicating that groups did not differ for the RGC counts in the OHT and Non-OHT eyes (FIG. 3). As a secondary analysis, FG-Labeled RGC counts per mm² were compared between the Non-OHT and the OHT retinas for each group using a paired-t test (two-tailed). Group 2 (vehicle topical; P=0.047) and Group 4 (0.25% Laquinimod topical; P=0.033) had fewer RGCs in the OHT eye compared to the Non-OHT eyes. The number of RGCs in Group 3 (4% Laquinimod topical; P=0.071) and Group 5 (0.1% Laquinimod oral; P=0.075) were statistically marginally different between the OHT and Non-OHT eyes. However, in Group (1% Laquinimod topical), there was no statistically significant difference in the RGC counts between the OHT and Non-OHT eyes (P=0.189). This suggests that the daily topical application of 1% Laquinimod may be neuroprotective for the RGC.
6. Optic Nerve (ON) Injury Grades: Mean ON Injury Grades (Mean±SD) for the Non-OHT eyes were 1.1±0.1, 1.2±0.2, 1.2±0.2, 1.2±0.3 and 1.2±0.2 for Groups 1, 2, 3, 4 and 5, respectively. Mean Injury Grades (Mean±SD) for the OHT optic nerves were 2.5±1.3, 3.0±1.6, 3.0±1.5, 2.8±1.3 and 2.7±1.5 for Groups 1, 2, 3, 4, and 5, respectively. A Two-Way ANOVA on the Mean ON injury grades in the OHT and Non-OHT (control) eyes versus groups did not show a significant eye versus group interaction (P=0.98), indicating that groups did not differ for the injury in the OHT and Non-OHT optic nerves (FIG. 4). As a secondary analysis, Mean ON Injury Grades were compared between the Non-OHT and the OHT optic nerves for each group using two-tailed paired-t tests. The Mean ON injury grades were significantly greater in the OHT eyes compared to the Non-OHT control eyes in all groups (P<0.05).
7. Ophthalmic Examinations (OEs): At baseline ocular examinations prior to initial dose, there were no ocular abnormalities observed in either eye in any of the animals included in the study. During the post-dose OEs, there were no ocular problems observed in any of the animals in the Non-OHT eyes. However, there were some ocular abnormalities observed in the OHT eyes due to the glaucoma-model creation procedures (hypertonic saline injection surgeries into the episcleral veins) in all groups. These abnormalities included minor congestion and chemosis (swelling) in the conjunctiva, minor corneal opacities, lack of sufficient dilation of the pupil after application of the pupil-dilating agent Tropicamide, slight opacities in the lens and presence of free-floating iris pigments in front of the lens in the OHT eyes. The post-HSI ocular findings in the OHT eyes were observed in all groups with a similar occurrence and were not test articlerelated.

### CONCLUSION

The study involved daily dosing of five groups for approximately six weeks: Group 1 (1% Laquinimod topical), Group 2 (Vehicle topical), Group 3 (4% Laquinimod topical), Group 4 (0.25% Laquinimod topical) and Group 5 (0.1% Laquinimod oral). The analyses for the RGC counts in Group 1 animals suggested a trend towards neuroprotection following daily topical application of 1% Laquinimod.

### Example 2: Assessment of Efficacy of Laquinimod for Treating Patients Afflicted with Glaucoma

Periodic (e.g., daily or twice daily) administration of laquinimod (oral or topical) is effective in treating glaucoma human patients. Periodic (e.g., daily or twice daily) administration of laquinimod (oral or topical) is effective to reduce a glaucoma-associated symptom in the subject.

A laquinimod composition as described herein is administered systematically or locally to the eye of a subject suffering from glaucoma. The administration of the composition is effective to treat the subject suffering from glaucoma. The administration of the composition is also effective to reduce a glaucoma-associated symptom of glaucoma in the subject. The administration of the composition is also effective to reduce intraocular pressure in the subject. The administration of the composition is effective to reduce RGC damage and/or RGC loss, and improve RGC viability in the subject.

### REFERENCES

1. “Glaucoma”, Merck Manual of Diagnosis and Therapy (1999), Merck Research Laboratories, (Whitehouse Station, N.J.), 733-738.
2. Alward, “Medical Management of Glaucoma”, N Eng J Med, 1998; 339:1298-1307).
3. Bakalash et al., “Resistance of Retinal Ganglion Cells to an Increase in Intraocular Pressure is Immune-dependent”, Invest Ophthalmol Vis Sci 2002; 43:2648-2653.
4. Brod et al. (2000) Annals of Neurology, 47:127-131.
5. Brück et al., (2011) “Insight into the mechanism of laquinimod action”, J. Neurol Sci. 306:173-179.
6. Cepurna et al. (2000) “Patterns of Intraocular Pressure Elevation After Aqueous Humor Outflow Obstruction in Rats.” Invest Ophthalmol Vis Sci. 41(6) (May 2000):1380-5.
7. Coleman “Glaucoma”, Lancet, 1999; 354:1803-1810.
8. Draize, J. H. (1965) “Appraisal of the Safety of Chemicals in Foods, Drugs, and Cosmetics.” Association of Food and Drug Officials of the United States, Austin, Tex., 1965. 36-45.
9. Farkas et al., “Apoptosis, Neuroprotection and Retinal Ganglion Cell Death: An Overview”, Int Ophthalmol Clin 2001; 41:111-130.
10. Freireich et al. (1966) “Quantitative comparison to toxicity of anticancer agents in mouse, rat, hamster, dog, monkey and man.” Cancer Chemother Rep, 50:219-244.
11. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 <http://www.fda.gov/cber/gdlns/metabol.pdf>.
12. Gurevich et al. (2010) “Laquinimod suppress antigen presentation in relapsing-remitting multiple sclerosis: invitro high-throughput gene expression study.” J Neuroimmunol. 2010 Apr. 15; 221(1-2):87-94. Epub 2010 Mar. 27.
13. Hla et al. (2001) “Lysophospholipids—receptor revelations.” Science, 294(5548):1875-8.
14. Horga and Montalban Jun. 4, 2008; Expert Rev Neurother., 2008; 8(5):699-714.
15. ISO/IEC 17025, 2005. General Requirements for the Competence of Testing and Calibration Laboratories.
16. Kipnis et al., “T Cell Immunity To Copolymer 1 Confers Neuroprotection On The Damaged Optic Nerve: Possible Therapy For Optic Neuropathies”, Proc Natl Acad Sci 2000; 97:7446-7451.
17. Kleinschmidt-DeMasters et al. (2005) New England Journal of Medicine, 353:369-379.
18. Langer-Gould et al. (2005) New England Journal of Medicine, 353:369-379.
19. McDonald, and Shadduck (1983). Eye Irritation in Dermatotoxicology (2nd Ed.). Edited by Marzulli F. N. Hemishpere Publishing Corp., New York, N.Y.
20. Medeiros et al., “Medical Backgrounders: Glaucoma”, Drugs of Today 2002; 38:563-570.
21. National MS Society Website, retrieved Jul. 10, 2012<http://www.nationalmssociety.org/ms-clinical-care-network/researchers/clinical-study-measures/index.aspx>
22. OECD 405, Organization for Economic Co-Operation and Development (OECD), Guidelines for the Testing of Chemicals, “Acute Eye Irritation/Corrosion”, adopted 24 Apr. 2002.
23. Ollivier F. J., et al. (2007) “Ophthalmic Examination and Diagnostics Part 1: The Eye Examination and Diagnostic Procedure” in Veterinary Ophthalmology, 4th Ed. by Gelatt, K. N. 2007, 438-483. Blackwell Publishing, Gainesville, Fla.
24. PCT International Application Publication No. WO 2007/0047863, published Apr. 26, 2007.
25. PCT International Application Publication No. WO 2007/0146248, published Dec. 21, 2007.
26. Polman et al. (2005) “Treatment with laquinimod reduces development of active MRI lesions in relapsing MS.” Neurology. 64:987-991.
27. Polman et al. (2011) “Diagnostic Criteria for Multiple Sclerosis: 2010 Revisions to the McDonald Criteria.” Ann Neural, 69:292-302.
28. Polman et al., (2005) “Diagnostic criteria for multiple sclerosis: 2005 revisions to the McDonald Criteria.” Annals of Neurology, 58(6):840-846.
29. Rudick et al. (1999) “Use of the brain parenchymal fraction to measure whole brain atrophy in relapsing-remitting MS: Multiple Sclerosis Collaborative Research Group”. Neurology. 53:1698-1704.
30. Runström et al. (2002) “Laquinimod (ABR-215062) a candidate drug for treatment of Multiple Sclerosis inhibits the development of experimental autoimmune encephalomyelitis in IFN-β knock-out mice,” (Abstract), Medicon Valley Academy, Malmoe, Sweden.
31. Sandberg-Wollheim et al. (2005) “48-week open safety study with high-dose oral laquinimod in patients.” Mult Scler. 11:S154 (Abstract).
32. U.S. Pat. No. 6,077,851, issued Jun. 20, 2000 (Bjork et al).
33. U.S. Pat. No. 7,589,208, issued Sep. 15, 2009 (Jansson et al).
34. Vollmer et al. (2008) “Glatiramer acetate after induction therapy with mitoxantrone in relapsing multiple sclerosis.” Multiple Sclerosis, 00:1-8.
35. Weinreb et al., “Is Neuroprotection a Viable Therapy for Glaucoma?” Arch Ophthalmol 1999; 117:1540-1544.
36. Yang et al. (2004) “Laquinimod (ABR-215062) suppresses the development of experimental autoimmune encephalomyelitis, modulates the Th1/Th2 balance and induces the Th3 cytokine TGF-β in Lewis rats.” J. Neuroimmunol. 156:3-9.

What is claimed is:

1. A method of treating a subject afflicted with glaucoma comprising administering to the subject an amount of laquinimod effective to treat the subject.

2. The method of claim 1, wherein the administration of laquinimod is effective to reduce or inhibit a symptom of the glaucoma in the subject.

3. The method of claim 2, wherein the symptom is retinal ganglion cell damage, retinal ganglion cell loss, or elevated intraocular pressure.

4. The method of any one of claims 1-3, wherein laquinimod is laquinimod sodium.

5. The method of any one of claims 1-4, wherein the route of administration of laquinimod is intraocular, periocular, ocular, oral, systemic or topical.

6. The method of any one of claims 1-5, wherein laquinimod is administered in the form of an aerosol, an inhalable powder, an injectable, a liquid, a gel, a solid, a capsule or a tablet.

7. The method of claim 6, wherein the concentration of laquinimod in the liquid or gel is 5-100 mg/ml solution, 20-100 mg/ml solution, 10-15 mg/ml solution, or 20-50 mg/ml solution.

8. The method of any one of claims 1-7, wherein laquinimod is administered periodically.

9. The method of claim 8, wherein laquinimod is administered daily.

10. The method of claim 8, wherein laquinimod is administered more often than once daily or less often than once daily.

11. The method of any one of claims 1-10, wherein the amount laquinimod administered is at least 0.2 mg/day and/or less than 0.6 mg/day.

12. The method of any one of claims 1-10, wherein the amount laquinimod administered is 0.03-600 mg/day, 0.1-40.0 mg/day, 0.1-2.5 mg/day, 0.25-2.0 mg/day, 0.5-1.2 mg/day, 0.25 mg/day, 0.3 mg/day, 0.5 mg/day, 0.6 mg/day, 1.0 mg/day, 1.2 mg/day, 1.5 mg/day or 2.0 mg/day.

13. The method of any one of claims 1-12, wherein the amount of laquinimod administered is 0.05-4.0 mg per administration, 0.05-2.0 mg per administration, 0.2-4.0 mg per administration, 0.2-2.0 mg per administration, about 0.1 mg per administration, or about 0.5 mg per administration.

14. The method of any one of claims 1-13, further comprising administration of a second agent for the treatment of glaucoma.

15. The method of claim 14, wherein the second agent is a β-adrenergic antagonist, adrenergic agonist, parasympathomimetic, prostaglandin-like analog, or carbonic anhydrase inhibitor.

16. The method of any one of claims 8-15, wherein the periodic administration of laquinimod continues for at least 3 days, more than 30 days, more than 42 days, 8 weeks or more, at least 12 weeks, at least 24 weeks, more than 24 weeks, or 6 months or more.

17. The method of any one of claims 1-16, wherein the subject is a human patient.

18. A method of treating a subject suffering from retinal ganglion cell loss or retinal ganglion cell damage, or of reducing retinal ganglion cell loss or damage in a subject, comprising administering to the subject an amount of laquinimod effective to reduce retinal ganglion cell loss or retinal ganglion cell damage in the subject.

19. A method of treating a subject suffering from elevated intraocular pressure, or of reducing intraocular pressure in a subject, comprising administering to the subject an amount of laquinimod effective to reduce intraocular pressure in the subject.

20. A package comprising:
a) a pharmaceutical composition comprising an amount of laquinimod; and
b) instruction for use of the pharmaceutical composition to treat a subject afflicted with glaucoma.

21. The package of claim 20, wherein the package comprises a second pharmaceutical composition comprising an amount of a second agent for the treatment of glaucoma.

22. The package of claim 21, wherein the second agent is a β-adrenergic antagonist, adrenergic agonist, parasympathomimetic, prostaglandin-like analog, or carbonic anhydrase inhibitor.

23. The package of any one of claims 20-22, wherein the pharmaceutical composition is the form of an aerosol, an inhalable powder, an injectable, a liquid, a gel, a solid, a capsule or a tablet.

24. The package of claim 23, wherein the concentration of laquinimod in the liquid or gel is 5-100 mg/ml solution, 20-100 mg/ml solution, 10-15 mg/ml solution or 20-50 mg/ml solution.

25. The package of claim 23, wherein the tablets are coated with a coating which inhibits oxygen from contacting the core.

26. The package of claim 25, wherein the coating comprises a cellulosic polymer, a detackifier, a gloss enhancer, or pigment.

27. The package of any one of claims 20-26, wherein the pharmaceutical composition further comprises mannitol, an alkalinizing agent, an oxidation reducing agent, a lubricant, a filler, and/or a desiccant.

28. The package of claim 27, wherein the alkalinizing agent is meglumine.

29. The package of any one of claims 20-27, wherein the pharmaceutical composition is stable and free of an alkalinizing agent or an oxidation reducing agent, preferably the pharmaceutical composition is free of an alkalinizing agent and free of an oxidation reducing agent.

30. The package of any one of claims 20-29, wherein the pharmaceutical composition is stable and free of disintegrant.

31. The package of claim 27, wherein the lubricant is present in the pharmaceutical composition as solid particles.

32. The package of claim 27 or 31, wherein the lubricant is sodium stearyl fumarate or magnesium stearate.

33. The package of claim 27, wherein the filler is present in the pharmaceutical composition as solid particles.

34. The package of claim 27 or 33, wherein the filler is lactose, lactose monohydrate, starch, isomalt, mannitol, sodium starch glycolate, sorbitol, lactose spray dried, lactose anhydrouse, or a combination thereof.

35. The package of claim 27, wherein the desiccant is silica gel.

36. The package of any one of claims 20-35, wherein the pharmaceutical composition is stable and has a moisture content of no more than 4%.

37. The package of any one of claims 20-36, wherein laquinimod is present in the pharmaceutical composition as solid particles.

38. The package of any one of claims 20-37, wherein the package is a sealed packaging having a moisture permeability of not more than 15 mg/day per liter or a blister pack having a maximum moisture permeability of not more than 0.005 mg/day.

39. The package of claim 38, wherein the sealed package is a bottle or comprises an HDPE bottle, preferably the bottle is closed with a heat induction liner.

40. The package of claim 38 or 39, wherein the sealed package comprises an oxygen absorbing agent, which oxygen absorbing agent is preferably iron.

41. The package of any one of claims 20-40, wherein the amount of laquinimod in the pharmaceutical composition is at least 0.2 mg, less than 0.6 mg, 0.1-40.0 mg, 0.03-600 mg, 0.1-2.5 mg, 0.25-2.0 mg, 0.5-1.2 mg, 0.25 mg, 0.3 mg, 0.5 mg, 0.6 mg, 1.0 mg, 1.2 mg, 1.5 mg, or 2.0 mg.

42. The package of any one of claims 20-41, wherein the pharmaceutical composition comprises unit doses of laquinimod of 0.05-4.0 mg, 0.05-2.0 mg, 0.2-4.0 mg, 0.2-2.0 mg, about 0.1 mg, or about 0.5 mg.

43. The package of any one of claims 20-42, wherein the pharmaceutical composition is formulated for intraocular, periocular, ocular, oral, systemic or topical administration.

44. The package of any one of claims 20-43, for use in treating a subject afflicted with glaucoma.

45. A therapeutic package for dispensing to, or for use in dispensing to, a subject afflicted with glaucoma, which comprises:
a) one or more unit doses, each such unit dose comprising an amount of laquinimod thereof, wherein the amount of said laquinimod in said unit dose is effective, upon administration to said subject, to treat the subject, and
b) a finished pharmaceutical container therefor, said container containing said unit dose or unit doses, said container further containing or comprising labeling directing the use of said package in the treatment of said subject.

46. The therapeutic package of claim 45, wherein the package comprises a second pharmaceutical composition comprising an amount of a second agent for the treatment of glaucoma.

47. The therapeutic package of claim 46, wherein the second agent is a β-adrenergic antagonist, adrenergic agonist, parasympathomimetic, prostaglandin-like analog, or carbonic anhydrase inhibitor.

48. A pharmaceutical composition comprising an amount of laquinimod for use in treating a subject afflicted glaucoma.

49. The pharmaceutical composition of claim 48, comprising an amount of a second agent for the treatment of glaucoma.

50. The pharmaceutical composition of claim 49, wherein the second agent is a β-adrenergic antagonist, adrenergic agonist, parasympathomimetic, prostaglandin-like analog, or carbonic anhydrase inhibitor.

51. The pharmaceutical composition of any one of claims 48-50, in the form of an aerosol, an inhalable powder, an injectable, a liquid, a gel, a solid, a capsule or a tablet.

52. The pharmaceutical composition of claim 51, wherein the concentration of laquinimod in the liquid or gel is 5-100 mg/ml solution, 20-100 mg/ml solution, 10-15 mg/ml solution or 20-50 mg/ml solution.

53. The pharmaceutical composition of claim 51, comprising a unit dose of 10 μL of an aqueous pharmaceutical solution which contains in solution at least 0.2 mg laquinimod.

54. The pharmaceutical composition of any one of claims 48-53, wherein laquinimod is laquinimod sodium.

55. The pharmaceutical composition of claim 51, wherein the tablets are coated with a coating which inhibits oxygen from contacting the core.

56. The pharmaceutical composition of claim 55, wherein the coating comprises a cellulosic polymer, a detackifier, a gloss enhancer, or pigment.

57. The pharmaceutical composition of any one of claims 48-56, further comprising mannitol, an alkalinizing agent, an oxidation reducing agent, a lubricant and/or a filler.

58. The pharmaceutical composition of claim 57, wherein the alkalinizing agent is meglumine.

59. The pharmaceutical composition of any one of claims 48-57, which is free of an alkalinizing agent or an oxidation reducing agent, preferably the pharmaceutical composition is free of an alkalinizing agent and free of an oxidation reducing agent.

60. The pharmaceutical composition of any one of claims 48-59, which is stable and free of disintegrant.

61. The pharmaceutical composition of claim 57, wherein the lubricant is present in the pharmaceutical composition as solid particles.

62. The pharmaceutical composition of claim 57 or 61, wherein the lubricant is sodium stearyl fumarate or magnesium stearate.

63. The pharmaceutical composition of claim 57, wherein the filler is present in the pharmaceutical composition as solid particles.

64. The pharmaceutical composition of claim 57 or 63, wherein the filler is lactose, lactose monohydrate, starch, isomalt, mannitol, sodium starch glycolate, sorbitol, lactose spray dried, lactose anhydrouse, or a combination thereof.

65. The pharmaceutical composition of any one of claims 48-64, wherein the amount of laquinimod is at least 0.2 mg, less than 0.6 mg, 0.1-40.0 mg, 0.03-600 mg, 0.1-2.5 mg, 0.25-2.0 mg, 0.5-1.2 mg, 0.25 mg, 0.3 mg, 0.5 mg, 0.6 mg, 1.0 mg, 1.2 mg, 1.5 mg, or 2.0 mg.

66. The pharmaceutical composition of any one of claims 48-65, comprising unit doses of laquinimod of 0.05-4.0 mg, 0.05-2.0 mg, 0.2-4.0 mg, 0.2-2.0 mg, about 0.1 mg, or about 0.5 mg.

67. The pharmaceutical composition of any one of claims 48-66, formulated for intraocular, periocular, ocular, oral, systemic or topical administration.

68. The pharmaceutical composition of any one of claims 48-67, for use in treating a subject afflicted with glaucoma.

69. A pharmaceutical composition in unit dosage form, useful in treating a subject afflicted with glaucoma, which comprises an amount of laquinimod; which amount of said laquinimod in said composition is effective, upon administration to said subject of one or more of said unit dosage forms of said composition, to treat the subject.

70. A package comprising:
a) a pharmaceutical composition of any one of claims 48-69; and
b) instruction for use of the pharmaceutical composition to treat a subject afflicted with glaucoma.

71. Laquinimod for the manufacture of a medicament for use in treating a subject afflicted glaucoma.
